(19) United States
(12) Patent Application Publication
(10) Pub. No.: US 2024/0284933 A1
(43) Pub. Date: Aug. 29, 2024

(54) RAW PLANT-BASED MEAT FROM PLANT-BASED MUSCLE FIBERS

(71) Applicant: Demolish Foods Inc., Niwot, CO (US)

(72) Inventors: Sandeep Sibal, Mumbai (IN); Ravali Amba, Secunderabad (IN); Amrita Behera, Odisha (IN); Prativa Das, Navi Mumbai (IN); Dhanesh Alagarsamy, Madurai (IN); Ajay Kumar Yadav, Rajasthan (IN); Raghavi Rao Kodati, Hyderabad (IN); Shirin Fatima, New Delhi (IN); Emma Dolan, Kirkland, WA (US); Subramoni Hariharan, Mumbai (IN)

(73) Assignee: Demolish Foods Inc., Niwot, CO (US)

(21) Appl. No.: 18/583,924

(22) Filed: Feb. 22, 2024

Related U.S. Application Data
(60) Provisional application No. 63/447,703, filed on Feb. 23, 2023.

(51) Int. Cl. A23J 1/00; A23P 20/18; A23P 30/10; C12N 5/04

(52) U.S. Cl. CPC A23J 1/006; A23P 20/18; A23P 30/10; C12N 5/04; C12N 2523/00

(57) ABSTRACT

Whole-cut imitators are disclosed mimicking animal muscles that have a basic structure that is common to most if not all animal species that are consumed for meat. The animal muscle mimics are made from plant proteins and other components that comprise whole-cuts that are in turn comprised of muscle fascicles, which in turn are made of plant-based muscle fibers. The muscles, fascicles and plant-based muscle fibers are bound together by extra-cellular matrix, (ECM), of which there are three kinds, a first ECM that binds plant-based muscle fibers together that is called the endomysium, a second ECM that coats and binds fascicles that is called the perimysium, and a third ECM that coats and binds muscles together that is called the epimysium.

### CROSS-REFERENCE TO RELATED APPLICATIONS

The present application claims priority under 35 USC 119(e) to U.S. Provisional Application No. 63/447,703 filed Feb. 23, 2023, the entire contents of which is incorporated by reference.

### FIELD OF THE INVENTION

The present invention relates to the area of plant-based whole-cut meats which are intended to mimic animal whole-cut meats. The specific invention is in the area of creating plant-based meats from plant-based muscle fibers that mimic animal meat fibers across a range of sensory, functional and nutritional properties.

### BACKGROUND OF THE INVENTION

Animal meat is a common source of protein, but concerns about its negative impact on the welfare of animals, the environment, climate change, and human health, and its inefficient usage of land and water resources have led to a demand for plant-based meat.

One of the biggest challenges in constructing plant-based meats that mimic animal meat is to recreate the complex species-specific structure of meat that is responsible for the unique texture, look and feel of meat, which is key to the experience of consuming meat.

Over the years, six major approaches have been invented and developed to recreate meat texture using plant proteins:
1. High Moisture Extrusion (HME)
2. Shear Cell Technology (SCT)
3. Freeze Structuring (FS)
4. Electro-spinning (ES)
5. Wet spinning (WS)
6. 3D Printing (3DP)

HME, SCT and FS are macro top-down approaches that directly produce meat from a mass of plant proteins and are generally considered to be limited in their ability to create 10 cm long and sub-100 micron thin muscle-like fibers to recreate whole-cuts of meat. They also require heat treatment of plant proteins (above 100° C. for HME and SCT) or low temperatures (below 0° C. for FS) that make them energy inefficient.

ES, WS and 3DP are micro bottom-up approaches that create microscopic elements that are then in turn assembled to create whole cuts of meat. Electro-spinning creates muscle fiber like structures for a certain class of proteins but is considered too low in throughput to be economical. Wet spinning creates muscle fiber like structures by extruding plant protein liquids into acid/alkaline baths that cause considerable waste and are also difficult to scale. Finally, 3DP is an additive technique that deposits sub-mm amounts of plant proteins to create structure but is not well suited to create 10 cm long and sub-100 micron thin muscle-like fibers that are sufficient in strength and length.

### BRIEF SUMMARY OF THE INVENTION

The present invention relates to mimicking whole-cuts made up of animal muscles that have a basic structure that is common to most if not all animal species that are consumed for meat. In an embodiment, the present invention relates to animal muscles made from plant proteins and other components that comprise whole-cuts that are in turn comprised of muscle fascicles, which in turn are made of plant-based muscle fibers. The muscles, fascicles and fibers are bound together by extra-cellular matrix, (ECM), which is of three types, a first ECM that binds plant-based muscle fibers together that is called the endomysium, a second ECM that coats and binds fascicles that is called the perimysium, and a third ECM that coats and binds muscles together that is called the epimysium.

### BRIEF DESCRIPTION OF THE SEVERAL VIEWS OF THE DRAWING

FIG. 1 depicts a spider chart showing desired mechanical properties and functional properties that one is trying to attain in a plant-based meat mimic.

FIG. 2 shows an example slice of a raw plant-based chicken muscle made using the technique of the present invention.

FIG. 3 shows a flow chart showing the process of the present invention to make plant-based meat.

FIG. 4 shows an example muscle fiber that is coated using the endomysium wherein part of the fiber has its endomysium removed to give an idea of the thickness of the naked plant-based muscle fiber using a micron ruler.

FIG. 5 shows a schematic representation showing how muscle fascicles are assembled and shaped into muscles and then into a whole-cut chicken breast using the methods of the present invention.

### DETAILED DESCRIPTION OF THE INVENTION

Whole-cuts made up of animal muscles have a basic structure that is common to most if not all animal species that are consumed for meat. Animal muscles that comprise whole-cuts are in turn comprised of muscle fascicles, which in turn are made of muscle fibers. Muscles, fascicles and fibers are bound together by an extra-cellular matrix (ECM). There are three main extra-cellular matrix parts of muscle whole cuts. The ECM which coats and binds muscle fibers together is called the endomysium. The ECM that coats and binds fascicles is called the perimysium. The ECM that coats and binds muscles together is called the epimysium.

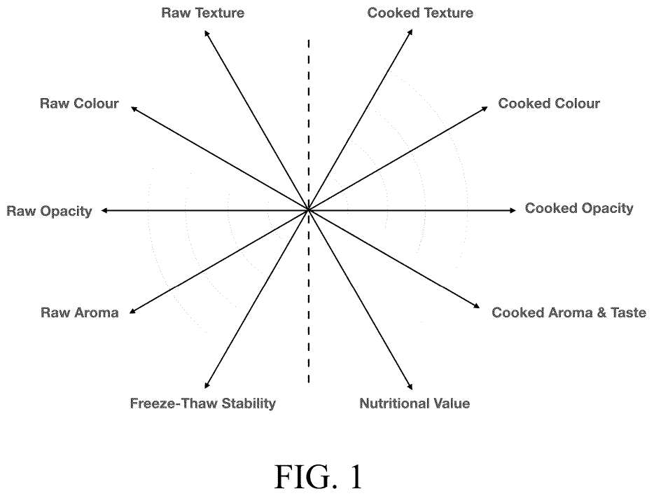

The fibers, fascicles, muscles and ECM (of all 3 types), play a crucial role in determining the mechanical properties and functional performance of the meat including ten of the key mechanical and functional properties depicted in FIG. 1. These mechanical and functional properties are a substantial part of what gives the meat the taste and texture that one typically finds in meats.

### Desirable Properties of Whole-Cut Meats:

In an embodiment, novel plant-based muscle fibers, are assembled and bound together using (a) plant-based ECM(s) that mimic(s) the animal ECMs (i.e., the endomysium, perimysium and epimysium). The closer that one is able to approach the structure and the mechanical and functional properties of meat, the closer one will be to attain the taste, texture, mouthfeel, look and feel, aroma and flavors of meat.

In an embodiment, the three types of plant-based ECMs initially exist in a low-viscosity liquid form allowing for ease of application to the plant-based muscle fibers/fascicles/muscles. By low-viscosity, it is meant that a viscosity is present that allows the plant-based muscle fibers/fascicles/muscles to be coated without gumming up a spray coater if using a spray coater or leaves a thin coat of ECM when being dip coated or dunk coated while allowing the fibers/fascicles/muscles to be evenly and completely coated. The visosity of endomysium is in the range of 0.9 to 1.5 cP. The viscosity of perimysium is in the range of 400-800 cP. Upon application, the plant-based ECMs undergo delayed gelation with no stimulus, or in a variant undergo gelation in the presence of a stimulus like ionic and/or enzymatic crosslinking agents etc. Upon gelation, the ECM forms a cohesive material that adheres to the plant-based muscle fibers/fascicles/muscles through crosslinking. This process forms a raw plant-based whole-cut muscle that mimics the mechanical and functional properties of raw animal muscle, and replicates cooked animal muscle when cooked.

In an embodiment, the ECMs may be derived from various plant sources including but not limited to legumes, cereals, oilseeds, and pseudocereals. The approach of the present invention in creating the 3 types of ECMs for plant-based muscle fibers, fascicles is more energy-efficient and cost-effective because these various ECMs are created at room temperature and it avoids using techniques that require high heat and/or freeze-thawing.

In an embodiment, by utilizing a bottom-up approach of structuring plant-based muscles by using plant-based muscle fibers that are separate, well organized and laid out, it is easy to differentiate between the application of endomysium, perimysium, and epimysium; and mimic their thickness and their functional impact on the meat. The precise approach of the present invention results in an improved mouthfeel for whole-cuts which more closely mimics natural meat.

In an embodiment, the approach of the present invention relates to creating ECMs for plant-based muscle fibers that does not involve the use of high heat. Therefore, the manufactured product of the instant invention achieves a raw appearance and texture for the resulting plant-based meat product that is heretofore unsurpassed.

In an embodiment, the ECMs that are manufactured are designed for delayed gelation and initially have low viscosity, and they can be uniformly applied and produce a thin layer of variable thickness on plant-based muscle fibers/fascicles/muscles using mature techniques like dip coating or spray coating.

In an embodiment, because the approach of the present invention involves creating plant-based muscle fascicles using plant-based muscle fibers, it allows for precise control over the number of plant-based muscle fibers in each fascicle as well as the number of fascicles in a muscle of a given size. Moreover, the approach allows bending and shaping the fascicles, thereby allowing the present invention to achieve a precise match to the look and feel of whole cuts as well as other properties of whole-cuts.

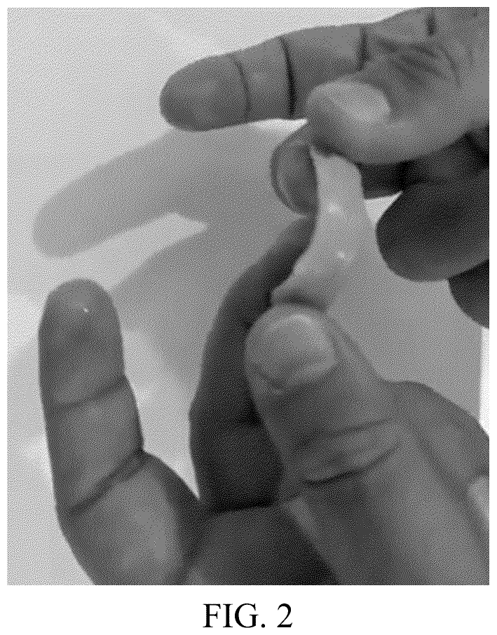

In an embodiment, FIG. 2 shows an example slice of a raw plant-based chicken muscle made using the technique of the present invention. The slice is comprised of tens of muscle fascicles and thousands of plant-based muscle fibers, which accurately mimics the look and feel of chicken muscle fascicle

### Process Flow Chart:

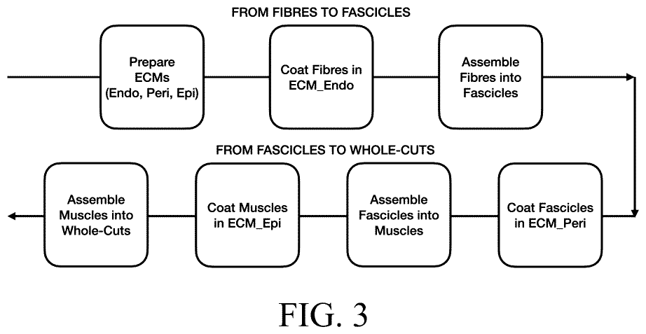

The flow chart that is shown in FIG. 3 shows the sequence of steps involved in the production of the plant-based meat of the present invention. This process can be broadly categorized into two parts:
1. From plant-based muscle fibers to fascicles
2. From fascicles to whole-cuts

### From Plant-Based Muscle Fibers to Fascicles

In an embodiment, the binding of plant-based muscle fibers using endomysium results in the formation of plant-based muscle fascicles. The properties of the endomysium and its interaction with the plant-based muscle fibers in conjunction with the properties of the plant-based muscle fibers play a crucial role in determining the properties of the species-specific muscle fascicle that is being imitated.

One of the challenges in creating the endomysium is being able to achieve low viscosity pre-gelation to allow easy and scalable application of the endomysium on plant-based muscle fibers, with the desired functional and mechanical properties of the endomysium while at the same time, having an endomysium that adheres well to plant-based muscle fibers and to each other. In an embodiment, the goal is to achieve similar properties to that of animal endomysium upon its gelation.

In an embodiment, after application of the endomysium onto the plant-based muscle fibers, the plant-based muscle fibers can then be shaped into muscle fascicles. The process of forming plant-based muscle fibers into fascicles comprises the following steps:

a. Prepare ECMs (Endomysium, Perimysium, Epimysium):

In an embodiment, to make a low-viscosity pre-gelled plant-based ECM, proteins that have both optimal gel strength and solubility are used along with a crosslinking agent that can bind the proteins in the plant-based muscle fibers to the amine groups present in the ECM. In a variant, the ECM might be solely composed of crosslinkers alone. In another variant, the ECM might also comprise glycosaminoglycans produced through fermentation techniques using genetically engineered or naturally occurring bacteria or yeast. In yet another variant, ECM might have a random distribution of fat deposits that may melt and weaken and loosen the connective tissues on cooking to mimic the fat distribution seen in animal muscle ECM.

In a variation, the ECM might include components that support the creation of a double network hydrogel. It is generally understood that double network hydrogels have better mechanical properties than single network hydrogels. In one embodiment, the first network may be composed of proteins bound together using a non-ionic crosslinker while the second network may be composed of a material that forms a gel network in presence of ionic crosslinkers. In a variation, the first network may comprise a material that forms a gel network in the presence of ionic crosslinkers and the second network may comprise proteins bound together using non-ionic crosslinkers. Double network hydrogels have several benefits, including, high mechanical strength, and high toughness, which means they are more resistant to fracture and can be stretched without breaking relative to the single network hydrogels. The improved mechanical properties of the ECM hydrogel as disclosed herein may allow the plant-based meat to mimic the texture and malleability of animal meat more closely, making it possible to tenderize the assembled plant-based meat using a mallet or similar techniques. Accordingly, the process of the present invention helps address some of the sensory and textural issues associated with plant-based meats. Accordingly, these plant-based meats should be more appealing to consumers who prefer the texture of animal meat.

In an embodiment, the ECM might be frozen and thawed after gelation using a blast freezer, plate freezer, proton freezer or any other freezing technique to help increase the mechanical properties of ECM hydrogel. The rate of freezing, freezing time and temperature can be optimized to achieve the desired amount of increase in mechanical strength, toughness and elasticity. Accordingly, this technique can be used to achieve a plant-based meat that more closely mimics true meat.

In an embodiment, a description of the base formulations for ECM and the factors that were taken into consideration while choosing the composing elements has been provided above. In the subsequent sections, the details of the three types of ECM and how they can be made are discussed.

### Endomysium:

In an embodiment, endomysium typically remains heat stable up to 100° C., suggesting that its contribution to texture is primarily through adhesion. Post cooking, the endomysium's adhesion remains at a similar level. Therefore, in one embodiment, the previously described ECM variant that solely uses protein-protein cross-linkers can be used to create the endomysium to closely replicate the animal endomysium's functional properties. In a variation, the endomysium contains randomly distributed fat deposits added to match the target muscle of the target species being mimicked.

### Perimysium:

In an embodiment, perimysium makes up more than 90% of the extracellular matrix (ECM) in most muscles and contributes significantly to the stiffness of raw and cooked whole-cut muscle. It undergoes more heat-induced changes than the endomysium does and the connection between the perimysium and fascicles weakens on cooking. Thus, in order to replicate the behavior of perimysium, an ECM consisting of plant proteins with optimal gelation properties when raw and cooked is used. Some plant proteins that may be used are soy proteins, potato proteins, pea proteins etc. In a variation, the perimysium may include a random distribution of fat deposits to match the fat deposit distribution in the target muscle of the target species being imitated.

### Epimysium:

In an embodiment, because the perimysium in animal muscles extends into the epimysium and forms an interconnected mesh together with the epimysium, the epimysium is typically made of the same material as perimysium and the epimysium can be made by using the plant proteins disclosed above. However, the fat content in epimysium is varied based on the target muscle of the target species so the fat deposits that are added should be adjusted accordingly.

### ECM Preparation Method:

a. In an embodiment, dry powder ingredients making up the ECM are primarily plant proteins (but in a variant they may include other additives) along with a crosslinker that forms bonds between the proteins of the ECM and proteins of the plant-based muscle fibers or fascicles. In a variation, the individual dry components are mixed in water until a uniform dispersion is achieved using one or more of a homogenizer, an ultrasonic homogenizer, a magnetic stirrer or other dispersion equipment. In a variation, solid fats can be introduced and dispersed as well.

b. Coat Plant-Based Muscle Fibers in Endomysium:

First, the plant-based muscle fibers are coated. In an embodiment, the plant-based muscle fibers may exist in one of the following orientations when they are being coated:
1. Horizontal: plant-based muscle fibers are laid flat in a container providing additional physical support to the plant-based muscle fibers when they are being coated but limiting the number of directly accessible surfaces for coating.
2. Vertical: plant-based muscle fibers are hung vertically exposing all surfaces directly for coating with endomysium. The vertical direction is generally preferable to the horizontal orientation as it allows for better coating ability.

In an embodiment, the low viscosity of the developed endomysium enables its application through a variety of coating techniques on the plant-based muscle fibers. The plant-based muscle fibers can be coated using one or more of the following different techniques:
1. Soaking: The collected plant-based muscle fibers can be oriented in a horizontal or vertical direction and may be left to soak in the endomysium liquid for a set period of time. Subsequently, the endomysium is allowed to drain off of the plant-based muscle fibers with or without the application of pressure.
2. Dip Coating: The collected plant-based muscle fibers are arranged in a vertical orientation and are dipped for a prescribed amount of time and are pulled out at a specific velocity to achieve a desired coating thickness without disturbing the orientation of the plant-based muscle fibers or causing entanglement.
3. Spray coating: The collected plant-based muscle fibers are oriented in either the horizontal or vertical direction and may be sprayed using an optimal mist flow rate producing atomizing nozzles. In a variation, these nozzles are used in order to deliver a precise amount of endomysium to the plant-based muscle fibers without disturbing the orientation of the plant-based muscle fibers or causing entanglement.

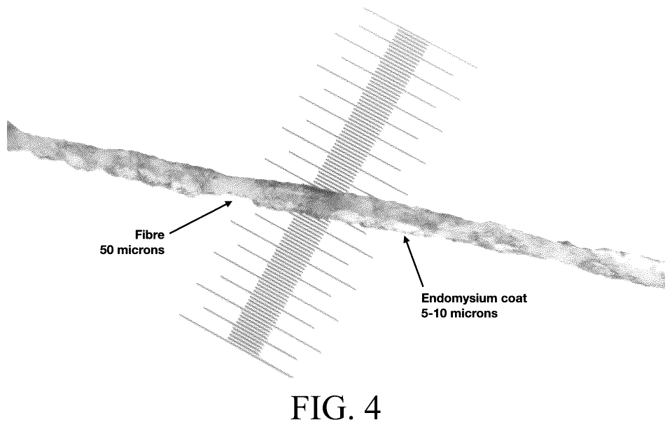

As shown in FIG. 4, an example plant-based muscle fiber that is coated using endomysium is shown. In FIG. 4, part of the plant-based muscle fiber has had its endomysium removed to show the thickness of the naked plant-based muscle fiber without endomysium.

c. Assemble Plant-Based Muscle Fibers into Fascicles:

In an embodiment, after being coated with endomysium, the plant-based muscle fibers are oriented vertically if they were previously in a horizontal orientation. Depending on the desired shape of the fascicles of the target muscle of the target species, the endomysium-coated plant-based muscle fibers may be assembled into fascicles using a shaper. The shaper can bring the plant-based muscle fibers together to form fascicles of various shapes, with such shapes including a cylindrical or pentagonal or sheet-like shape, among others. In one variation, the fascicles are formed using a shaper under vacuum conditions. In a variation, the fascicles are formed using a shaper using pressure. Alternatively, the plant-based muscle fibers may skip the shaper step and proceed directly to the application of perimysium before being shaped.

In an embodiment, the shaped fascicles may be induced to form crosslinks within the endomysium and/or between endomysium and plant-based muscle fibers by using one or more of the following procedures: setting an optimal temperature between 4° C. to 50° C. for gelation, or alternatively between about 10 to 40° C., or alternatively, between about 20 to 30° C., treating the fascicles in a salt bath for a set period, and/or exposing the plant-based muscle fibers to pressure as may be necessary. Alternatively, the plant-based muscle fibers may skip this crosslinking step entirely and proceed directly to the application of perimysium.

### From Fascicles to Muscles:

a. Coat Fascicles in Perimysium:

In an embodiment, the fascicles that are formed by assembling endomysium-coated plant-based muscle fibers or stand-alone endomysium coated plant-based muscle fibers from the previous step are hung in a vertically oriented manner (if they are not already in a vertical orientation). In a variation, they are then coated in perimysium in a similar manner to how the endomysium is coated on plant-based muscle fibers as described in the previous section. The excess perimysium is drained in a process similar to the draining process described in the previous step for endomysium with or without pressure, and/or with or without high-frequency low amplitude vibration in the horizontal plane.

b. Assemble Fascicles into Muscles:

In an embodiment, the perimysium coated fascicles are assembled together by first using a shaper to gather the fascicles into a desired shape such as that of the pectoralis major or the pectoralis minor of chicken, or alternatively, any other target muscles of a target species. The fascicles gathered into a muscle shape are induced to form crosslinks within the perimysium, between endomysium and perimysium, and between perimysium and exposed portions of plant-based muscle fibers by using one or more of the following options: an optimal temperature between 4° C. to 50° C. for gelation, or alternatively, about 10 to 40° C., or alternatively about 20 to 30° C., treating the fascicles in a salt bath for a set period, and/or pressure as may be necessary. The defined conditions may also induce crosslinking in the endomysium itself and/or between the endomysium and plant-based muscle fibers, if the crosslinking did not already occur in the previous step.

c. Coat Muscles in Epimysium:

In an embodiment, the muscles assembled in the previous step are coated in the epimysium using soaking or spraying. In a variation, in a subsequent step, the surplus epimysium may be allowed to drain out as described in previous sections.

d. Assemble Muscles into Whole-Cuts:

In an embodiment, the epimysium-coated muscles are assembled together, and in a final shaper step, they ae designed to resemble animal whole-cuts. The epimysium may be induced to form crosslinks within itself, with the epimysium of the neighboring muscle, with the perimysium, with any exposed endomysium and/or with any exposed plant-based muscle fibers using one or more of the following process conditions: an optimal temperature between 4° C. to 50° C. for gelation, or alternatively, about 10 to 40° C., or alternatively about 20 to 30° C., treating the fascicles in a salt bath for a set period, and/or pressure as may be necessary.

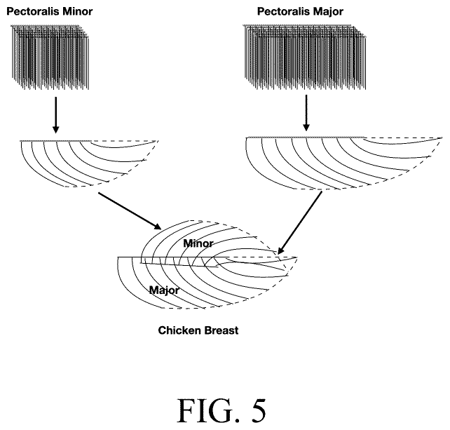

A schematic showing how muscle fascicles are assembled and shaped into muscles and then into a whole-cut chicken breast is shown in FIG. 5. The plant-based muscle fibers that have been coated with the various ECMs are oriented in the vertical direction and the pectoralis minor and the pectoralis major are generated separately. In a last step, the pectoralis minor and the pectoralis major are joined together, and shaped to make a shape similar to a chicken breast. Thus, not only does the plant-based meat have the texture, taste, aroma and other properties of the real chicken breast, but it also has the appearance of the real chicken breast.

The following references are incorporated by reference in their entireties for all purposes.
1. “Demolish Foods Debuts Breakthrough Plant Protein Muscle Fibers at Future Food Tech Summit—San Francisco,” 23 Mar. 2022, https://www.prnewswire.com/news-releases/demolish-foods-debuts-breakthrough-plant-protein-muscle-fibers-at-future-food-tech-summit---san-francisco-301508485.html
2. “Structuring processes for meat analogues,” Trends in Food Science & Technology, Volume 81, Pages 25-36, November 2018, https://doi.org/10.1016/j.tifs.2018.08.011

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A method of making raw plant-based meat, the method comprising:
a) Preparing a first extracellular matrix (ECM), a second ECM, and a third ECM;
b) Coating plant-based muscle fibers with the first ECM to generate ECM coated plant-based muscle fibers;
c) Assembling the ECM coated plant-based muscle fibers into fascicles;
d) Coating the fascicles with the second ECM to generate a muscle,
e) Coating the muscle with the third ECM to generate a coated muscle;
f) Assembling the coated muscle to generate the raw plant based meat.

2. The method of claim 1, wherein the first ECM, the second ECM, and the third ECM are an endomysium, a perimysium, and an epimysium, respectively.

3. The method of claim 2, wherein the method is performed at a temperature between about 4 and 50° C.

4. The method of claim 1, further comprising a step of adding crosslinkers.

5. The method of claim 1, further comprising a step of shaping one or more of the plant-based muscle fibers, fascicles, and/or muscles.

6. The method of claim 4, further comprising a step of shaping one or more of the plant-based muscle fibers, fascicles, and/or muscles.

7. The method of claim 1, wherein the coating is done by one or more of soaking, dip coating, and/or spray coating.

8. The method of claim 7, wherein the coating is done by orienting the plant-based muscle fibers or the muscles in a vertical direction.

9. The method of claim 2, wherein the endomysium, the perimysium, and the epimysium are made from plant proteins.

10. The method of claim 9, wherein any combination of the endomysium, the perimysium, and the epimysium comprise fat deposits.

11. The method of claim 6, wherein shaping is done under a vacuum.

12. The method of claim 7, wherein the endomysium, the perimysium, and/or the epimysium are of a low-viscosity.

13. The method of claim 12, wherein the endomysium, the perimysium, and/or the epimysium further comprise glycosaminoglycans produced through fermentation techniques.

14. The method of claim 8, wherein the coating is done by spray coating using an optimal mist flow rate.

15. A raw plant-based meat made by a method comprising:
a) Preparing a first extracellular matrix (ECM), a second ECM, and a third ECM;
b) Coating plant-based muscle fibers with the first ECM to generate ECM coated plant-based muscle fibers;
c) Assembling the ECM coated plant-based muscle fibers into fascicles;
d) Coating the fascicles with a second ECM to generate a muscle,
e) Coating the muscle with a third ECM to generate a coated muscle;
f) Assembling the coated muscle to generate the raw plant based meat.

16. The raw plant-based meat of claim 15, further comprising a method step of adding crosslinkers.

17. The raw plant-based meat of claim 15, further comprising a method step of shaping one or more of the plant-based muscle fibers, fascicles, and/or muscles.

18. The raw plant-based meat of claim 17, wherein the raw plant-based muscles are shaped into a shape of a chicken breast.

19. The raw plant-based meat of claim 15, wherein the raw plant-based meat further comprises fat deposits.

20. The raw plant-based meat of claim 15, further comprising one or more of method steps:
Setting a temperature between 4° C. to 50° C. for gelation, treating the fascicles in a salt bath for a set period, and/or pressure.
